(19) United States
(12) Patent Application Publication
(10) Pub. No.: US 2002/0034480 A1
(43) Pub. Date: Mar. 21, 2002

(54) Make-up compositions comprising a binder phase, and make-up methods

(76) Inventors: Sabine Grimm, Chatenay Malabry (FR); Anke Hadasch, Paris (FR); Nadia Terren, Bourg la Reine (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 09/860,566

(22) Filed: May 21, 2001

(30) Foreign Application Priority Data
May 19, 2000 (FR) 00 06451

(51) Int. Cl. A61K 7/021; A61K 7/025

(52) U.S. Cl. 424/63; 424/64

(57) ABSTRACT

A make-up composition comprising a pulverulent phase and a binder phase, wherein the binder phase comprises a dispersion of cubic gel particles and processes comprising such compositions.

The present invention relates to a make-up composition comprising a binder phase. These make-up compositions may be products for the complexion, such as foundations and powders, for example free, compact, pressed or cast powders, or foundations, and products which may act both as a powder and as a foundation, or make-up products for the body. These compositions may also be products for the lips, for instance, lipsticks, and products for the eyes, for instance, mascaras and eyeliners. They may be in the form of cast products, creams, powders, etc.

Make-up products may comprise a pulverulent phase comprising pigments and fillers and a dispersing, i.e., binder, phase comprising fatty substances. The dispersing (binder) phase may impart at least one of the following attributes to the make-up products: homogeneous dispersion of the pulverulent compounds, facilitation of pulverulent compounds' cohesion in the final composition, adjustment of the density of the finished product, provision of softness and emollient properties to the make-up product, and promotion of the make-up product's adhesion to the skin.

The development of dispersing or binder phases in make-up products may raise many difficulties.

For example, in the case of powders, the final composition should be sufficiently uniform and compact so as to have a good capacity to be taken up and to avoid fragmentation caused, for example, by impacts.

It is possible to use a mixture of mineral and plant oils combined with esters as a binder phase in powders. However, the products comprising these esters may lead to products lacking softness or poor dispersion of the pulverulent phase.

It is also possible to use silicone oils, which may provide at least one property chosen from slipperiness, softness and spreading ease, but staying power and impact resistance of the make-up may be adversely affected.

Additionally, certain perfluoro oils, for example, perfluoropolyethers, are known to provide softness to cosmetic compositions, such as powders. However, the product formulator is then often confronted with problems of insolubility of these oils in the usual hydrocarbon-based oils or silicone oils. This insolubility may lead to problems of instability of the composition. Moreover, compositions comprising perfluoro oils may have mediocre impact strength properties. Some of these oils may also have the drawback of poor dispersion of the pulverulent phase and can lead to relatively low color intensity for the pigments, possibly resulting in a “white effect”, which is not desirable, aesthetically speaking.

Make-up powders are thus products generally comprising a high content of pulverulent compounds. These products may be qualified as being of the “dry” or “oil” type. These products may impart sensations of tautness or dryness when they are applied to the skin. However, due to the high content of pulverulent compounds, it may be difficult to introduce large amounts of water into such products. The reason for this is that the water does not bind the particles, but rather, the water aggregates with the pulverulent compounds to form an unstable heterogeneous paste or mix. This paste is unsuitable to use as a make-up product since a measured amount of product cannot be taken up. Moreover, a powdered product made from a sticky paste or mix, may result in a plaster-like complexion, rather than the desired velvety appearance.

Another drawback to introducing water into make-up powders is that it is difficult to incorporate active agents, such as, hydrophilic active agents, into the powders and still maintain a stable composition.

Now, women increasingly wish to use make-up products which not only enhance their beauty but also give the skin a fresh sensation or a care effect.

In make-up products for the body, such as, for example, foundations, lipsticks, mascaras, and eyeliners, it is desirable to achieve a final composition that is homogeneous having well dispersed pulverulent compounds to ensure uniform application of make-up to the skin.

It is difficult to disperse pulverulent compounds satisfactorily in a foundation by primary means such as, for example, a spatula and an ordinary magnetic stirrer. One way to optimize the dispersion of these pulverulent compounds in make-up products, for example, is to first prepare a paste comprising pigments and fatty substances that are suitable for dispersing the pigments, and then to subsequently add the rest of the compounds of the composition. Another approach is to employ mixer-blender machines to efficiently disperse the pulverulent compounds with the aid of paddles or impellers.

Incorporation of water into foundations, mascaras, eyeliners, and lipsticks is known. Typically, such products may conventionally be in the forms of water-in-oil (W/O) and oil-in-water (O/W) emulsions. However, in such products, the pulverulent compounds, such as, pigments are not always optimally dispersed. Moreover, it is desirable to introduce into products, such as, foundations cosmetic-care active agents which are not oxidized by the pigments, to maintain a stable and homogeneous composition.

Thus, it would be useful to be able to prepare make-up products, for example, products for the complexion such as powders and foundations, comprising a binder phase which possesses at least one of the following properties: optimally disperses the pulverulent compounds, conveys active agents and gives the sensation of freshness on the skin.

The inventors have unexpectedly found that use of a composition comprising a dispersing, i.e., binder phase with cubic gel particles makes it possible to obtain make-up products which exhibit effective dispersion of pigments and affords fresh sensation and moisturization upon application.

One embodiment of the invention is thus a make-up composition comprising a pulverulent phase and a binder phase, wherein said binder phase comprises a dispersion of cubic gel particles.

The compositions thus obtained can bring about a pleasant sensation of freshness when applied. They can moisturize the skin and not give a sensation of tautness or dryness and they can effectively disperse the pigments. The composition can be homogeneous and may remain so even several hours after it has been applied to the skin.

In powder form, the compositions of the present invention, can exhibit effective staying power by minimizing the transfer and migration of the powder into the folds of the skin.

The present invention makes it possible to prepare foundation compositions in a simple manner. For example, it is possible to prepare a homogeneous foundation by placing only a small amount of dispersing, i.e., binder phase into the palm of the hand and then adding at least one pulverulent compound, such as, for example, pigments to the binder phase. By simply mixing the pigments together with a finger, a colored composition comprising well dispersed pigments is obtained. The make-up thus prepared can be homogeneous and uniform and may not form flakes on the face, as is the case when a conventional binder is used in the same way. These compositions, mixed in the palm of the hand, may be used directly by applying them to and spreading them on the skin like a standard foundation. Thus, any user who has at her disposal a bottle of binder phase according to the invention and a panel of pots comprising pulverants such as pigments, fillers, and active agents in pulverulent form, is capable of making her own made-to-measure foundation, as desired and in the amount desired.

The foundations according to the invention may exhibit effective staying power (no color change) and transparent matt effects.

Other make-up products for the body, such as, lipstick, mascara, and eyeliner compositions may also be prepared in the same manner as the previously described foundation compositions.

The compositions according to the invention may also have at least one of the following cosmetic properties: adequate skin adherence (adhere to the skin enough but not too much), softness and easy application.

The present invention also relates to a cosmetic process for treating (making up, or caring for) human keratinous materials such as skin and body, comprising application of the compositions as defined above to these materials.

According to the present invention, the term “keratinous materials” refers to skin, nails, hair, eyelashes, eyebrows, mucous membranes (inner edge of the lower eyelids), semi-mucous membranes (lips), and any other area of body and facial skin.

Another embodiment of the invention relates to a method of using at least one dispersion of cubic gel particles in a make-up composition comprising pulverulent compounds to improve the dispersion of the pulverulent compounds in the composition. The dispersion of cubic gel particles generally functions to aid dispersion of the pulverulent compounds in the composition.

The invention also relates to the use of at least one dispersion of cubic gel particles in a make-up composition to improve the moisturization of keratinous materials in the composition.

Another embodiment of the present invention relates to a make-up kit comprising a plurality of containers, wherein said plurality of containers comprises at least a first container and a second container, said first container contains a composition comprising at least one dispersion of cubic gel particles and said second container contains at least one pulverulent compound.

The compositions according to the invention may be advantageously used in the fields of make-up for and care of human keratinous materials, such as, skin and mucous membranes. Non-limiting examples of make-up products for the face and the skin are chosen from eye shadows, face powders, body powders, concealer products, foundations, make-up products for the body, lipsticks, mascaras, eyeliners, anti-sun products and self-tanning products.

Other characteristics, aspects and advantages of the present invention which may pertain to at least one embodiment of the present invention will become apparent upon reading the detailed description which follows.

The compositions according to the invention comprise a pulverulent phase which may comprise at least one pulverulent chosen from pigments, nacres, fillers, and flakes usually used in cosmetic compositions.

The term “pigment” may be understood to mean particles intended to color and optionally opacify the composition of the present invention. These pigments may be white or colored and made from mineral or organic origin. The pigments are insoluble in the medium of the compositions according to the invention.

The pigments may be present in an amount, for example, ranging from 0.05% to 80% by weight relative to the total weight of the composition, such as from 0.5% to 50%. The pigments may be white or colored and from mineral or organic origin. The pigments may also be of usual size or of nanometric size.

Non-limiting representatives of mineral pigments and nanopigments may be chosen from titanium dioxide, zirconium dioxide, cerium dioxide, zinc oxide, iron oxide, chromium oxide, nanotitaniums, nanozincs and ferric blue. Non-limiting representatives of organic pigments may be chosen from carbon black and lakes, such as, for example, calcium salts, barium salts, aluminium salts and zirconium salts, and acidic dyes, such as, for example, halo acid dyes, azo dyes and anthraquinone dyes.

The pigments may be coated with at least one compound chosen from silicone compounds such as, for example, polydimethylsiloxanes and polymers, such as polyethylenes. Mention may thus be made of the “SI oxides,” which are polymethylhydrogenosiloxane-coated pigments sold by the company MIYOSHI.

For the purposes of this invention, the term “filler” is understood to include colorless and white, lamellar and non-lamellar, particles of mineral or synthetic origin which may provide at least one of the following properties to the composition: body, rigidity, softness, a matt effect and uniformity.

The fillers, which may be present in the composition in an amount, for example, ranging from 0.05% to 99% by weight relative to the total weight of the composition may be lamellar and non-lamellar particles of mineral or synthetic origin. Non-limiting examples of fillers may be chosen from talc, mica, silica, kaolin, Nylon powder, poly-β-alanine powder, polyethylene powder, Teflon, lauroyllysine, starch, boron nitride, bismuth oxychloride, tetrafluoroethylene polymer powders, polymethyl methacrylate powders, polyurethane powders, polystyrene powders, polyester powders, synthetic hollow microspheres, such as, for example, Expancel (Nobel Industrie), microsponges, such as Polytrap (Dow Corning), silicone resin microbeads (for example Tospearls from the company Toshiba), zinc oxide, titanium oxide, zirconium oxide, cerium oxide, precipitated calcium carbonate, magnesium carbonate, magnesium hydrocarbonate, hydroxyapatite, hollow silica microspheres (silica beads from the company Maprecos), glass microcapsules, ceramic microcapsules, metal soaps derived from organic carboxylic acids comprising from 8 to 22 carbon atoms, such as from 12 to 18 carbon atoms, for example, zinc stearate, magnesium stearate, lithium stearate, zinc laurate and magnesium myristate.

The term “nacre” may be understood as meaning iridescent particles which reflect light.

The nacres may be present in the composition according to the invention, in an amount, for example, ranging from 0.05% to 80% by weight relative to the total weight of the composition, such as, for further example, in an amount ranging from 2% to 50% by weight. Non-limiting representatives of the nacres which may be used in the composition according to the invention, may be chosen from natural mother-of-pearl, mica coated with titanium oxide, mica coated with iron oxide, mica coated with natural pigment, mica coated with bismuth oxychloride, and colored titanium mica.

The compositions according to the invention may further comprise flakes.

The pulverulent phase may be present in the compositions according to the invention in an amount, for example, of at least 0.1% by weight relative to the total weight of the composition, such as, at least 2% by weight relative to the total weight of the composition.

The compositions according to the invention comprise a dispersing, i.e., binder phase. In one embodiment of the invention, this binder phase is a composition comprising an aqueous continuous phase, wherein cubic gel particles are dispersed in said aqueous continuous phase.

The term “cubic gel” used in accordance with the present invention denotes transparent gels which are isotropic in polarized light, and are in the form of cubic liquid crystal phases. The cubic liquid crystal phases are organized in a bipolar manner into separate hydrophilic and lipophilic domains forming a thermodynamically stable, three-dimensional network. Such an organization has been described, for example, in “La Recherche”, Vol. 23, pp. 306-315, March 1992 and in “Lipid Technology”, Vol. 2, No. 2, pp. 42-45, April 1990, all of which relating to such organizations are incorporated by reference herein. Depending on the arrangement of the hydrophilic and lipophilic domains, the cubic phase is said to be of normal or reverse type. The term “cubic gel” used in the present invention groups together gels having various types of cubic phases.

Non-limiting representative of such cubic gels are disclosed, for example, in EP 0 686 386 and EP 0 711 540, all of which relating to such cubic gels are incorporated by reference herein.

In one embodiment of the invention, the binder phase is an aqueous continuous phase comprising, or if there is a difference, formed from at least:

(i) 3,7,11,15-tetramethyl-1,2,3-hexadecanetriol, i.e., phytanetriol, wherein said compound is present in an amount ranging, for example, from 0.1% to 15% by weight relative to the total weight of the binder phase, and

(ii) at least one dispersing and stabilizing agent chosen from surface-active agents chosen from optionally saturated, linear and branched surface-active agents comprising fatty chains having from 8 to 22 carbon atoms, wherein said at least one dispersing and stabilizing agent is present in an amount ranging, for example, from 0.1% to 3% by weight relative to the total weight of said binder phase and further wherein said surface-active agents are water-soluble at room temperature.

In one embodiment of the invention, the binder phase comprises dispersed cubic gel particles comprising phytanetriol and an aqueous phase. The aqueous phase comprises at least one dispersing and stabilizing agent chosen from surface-active agents chosen from optionally saturated, linear and branched surface-active agents comprising fatty chains having, for example, from 8 to 22 carbon atoms.

According to one embodiment, phytanetriol is present in the composition according to the invention in an amount ranging from 0.5% to 10% by weight relative to the total weight of the binder phase.

The weight ratio of the phytanetriol to said dispersing and stabilizing agent, as defined above, ranges, for example, from 1:1 to 200:1, such as, for example, from 2:1 to 50:1.

Phytanetriol is a known compound which is sold, for example, under the name “Phytanetriol-63926®” by the company ROCHE.

In a further embodiment of the invention, the binder phase is chosen from compositions comprising:

(1) an aqueous phase, wherein said aqueous phase is present in an amount ranging, for example, from 60% to 98% by weight relative to the total weight of said composition; and

(2) an oily phase, wherein said oily phase is present in an amount ranging, for example, from 2% to 40% by weight relative to the total weight of said composition, and further wherein said oily phase is dispersed in said aqueous phase and is stabilized with the aid of cubic gel particles, said cubic gel particles comprise:

(i) at least one compound chosen from 3,7,11,15-tetramethyl-1,2,3-hexadecanetriol, i.e., phytanetriol, N-2-alkoxycarbonyl derivatives of N-methylglucamine, and unsaturated fatty acid monoglycerides, wherein said at least one compound is present in an amount ranging, for example, from 0.1% to 15% by weight relative to the total weight of the composition; and

(ii) at least one dispersing and stabilizing agent chosen from surface-active agents chosen from optionally saturated, linear and branched surface-active agents comprising fatty chains having, for example, 8 to 22 carbon atoms, wherein said at least one dispersing and stabilizing agent is present in an amount, for example, ranging from 0.05% to 3% by weight relative to the total weight of said composition and further wherein said surface-active agents are water-soluble at room temperature.

In one embodiment, the weight ratio of said cubic gel particles to said oily phase ranges, for example, from 0.02:1 to 1:1, such as, from 0.05:1 to 0.5:1.

According to another embodiment of the invention, the relative weight ratio of said cubic gel particles to said at least one dispersing and stabilizing agent ranges, for example, from 2:1 to 200:1, such as, for example, less than or equal to 50:1.

Non-limiting examples of N-2-alkoxycarbonyl derivatives of N-methylglucamine are chosen from at least one compound of formula (I):

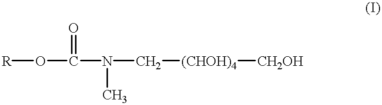

wherein R is chosen from branched (C₆-C₁₈) alkyl groups.

Non-limiting representatives of these derivatives may be chosen from N-2-hexyldecyloxycarbonyl-N-methylglucamine, N-2-ethylhexyloxycarbonyl-N-methylglucamine and N-2-butyloctyloxycarbonyl-N-methylglucamine.

A method for preparing the compounds of formula (I) is disclosed in EP-0 711 540, which is incorporated by reference herein.

According to one embodiment of the invention, the binder phase is chosen from compositions comprising:

(1) an aqueous phase, wherein said aqueous phase is present in an amount ranging from 60% to 98% by weight relative to the total weight of said composition; and

(2) an oily phase, wherein said oily phase is present in an amount ranging from 2% to 40% by weight relative to the total weight of said composition, and further wherein said oily phase is dispersed in said aqueous phase and is stabilized with the aid of cubic gel particles, said cubic gel particles comprise:

(i) a mixture comprising:

(a) phytanetriol in an amount ranging, for example, from 1% to 40% by weight relative to the total weight of said mixture; and

(b) at least one compound of formula (I):

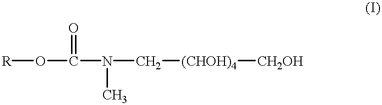

wherein R is chosen from branched (C₆-C₁₈) alkyl groups and said at least one compound of formula (I) is present in an amount ranging, for example, from 60% to 99% by weight relative to the total weight of said mixture; and

(ii) at least one dispersing and stabilizing agent chosen from surface-active agents chosen from optionally saturated, linear and branched surface-active agents comprising fatty chains having 8 to 22 carbon atoms, wherein said at least one dispersing and stabilizing agent is present in an amount ranging, for example, from 0.05% to 3% by weight relative to the total weight of said composition and further wherein said surface-active agents are water-soluble at room temperature.

According to one embodiment component (i) of said cubic gel particles defined above, may comprise, or if there is a difference, be formed from at least a mixture comprising:

(a) phytanetriol in an amount ranging from 10% to 30% by weight relative to the total weight of said mixture; and

(b) at least one compound of formula (I) in an amount ranging from 70% to 90% by weight relative to the total weight of said mixture.

Examples of unsaturated fatty acid monoglycerides are those chosen from (C₁₆-C₂₂) unsaturated fatty chains.

Non-limiting representatives of these monoglycerides may be chosen from glyceryl monooleate, glyceryl monoolein, glyceryl monolinoleate and glyceryl monolinolein.

It is possible to use, in the compositions according to the invention, mixtures comprising at least one compound chosen from unsaturated fatty acid monoglycerides, as defined above, and saturated fatty acid monoglycerides, the proportion of saturated fatty acid monoglycerides being, for example, lower than that of unsaturated fatty acid monoglycerides.

According to another embodiment of the invention, the binder phase is chosen from compositions comprising:

(1) an aqueous phase, wherein said aqueous phase is present in an amount ranging, for example, from 60% to 98% by weight relative to the total weight of said composition; and

(2) an oily phase, wherein said oily phase is present in an amount ranging, for example, from 2% to 40% by weight relative to the total weight of said composition, and further wherein said oily phase is dispersed in said aqueous phase and is stabilized with the aid of cubic gel particles, said cubic gel particles comprise:

(i) a mixture comprising:

(a) phytanetriol in an amount ranging, for example, from 1% to 50% by weight relative to the total weight of said mixture; and

(b) at least one unsaturated fatty acid monoglyceride present in an amount ranging, for example, from 50% to 99% by weight relative to the total weight of said mixture; and

(ii) at least one dispersing and stabilizing agent chosen from surface-active agents chosen from optionally saturated, linear and branched surface-active agents comprising fatty chains having 8 to 22 carbon atoms, wherein said at least one dispersing and stabilizing agent is present in an amount ranging, for example, from 0.05% to 3% by weight relative to the total weight of said composition and further wherein said surface-active agents are water-soluble at room temperature.

According to another embodiment of the invention, component (i) of said cubic gel particles defined above, may comprise, or if there is a difference, be formed from at least a mixture comprising:

(a) phytanetriol in an amount ranging, for example, from 10% to 30% by weight relative to the total weight of the mixture; and

(b) at least one unsaturated fatty acid monoglyceride in an amount ranging, for example, from 70% to 90% by weight relative to the total weight of the mixture.

Non-limiting examples of said at least one dispersing and stabilizing agent, defined above, are chosen from:

polyol alkyl ethers, polyol alkenyl ethers, polyol alkyl esters, and polyol alkenyl esters;

N-acylated amino acids and derivatives thereof;

peptides, wherein said peptides are N-acylated with at least one group chosen from alkyl groups and alkenyl groups, and salts thereof;

alkyl ethers, alkenyl ethers, alkyl ester sulfates and alkenyl ester sulfates, derivatives and salts thereof;

polyoxyethylenated alkyl fatty ethers, polyoxyethylenated alkenyl fatty ethers, polyoxyethylenated alkyl fatty esters, and polyoxyethylenated alkenyl fatty esters;

polyoxyethylenated alkyl carboxylic acids, polyoxyethylenated alkenyl carboxylic acids and salts thereof;

N-alkyl betaines, and N-alkenyl betaines; and

alkyltrimethylammonium, alkenyltrimethylammonium and salts thereof.

In the compounds listed above, the alkyl and alkenyl groups have 8 to 22 carbon atoms, and since “at least one” is used, mixtures are manifestly contemplated.

Non-limiting representatives of polyol alkyl ethers, polyol alkenyl ethers polyol alky esters and polyol alkenyl esters may comprise alkyl esters of sorbitan and alkenyl esters of sorbitan which are polyoxyethylenated with at least 20 ethylene oxide units, such as, for example, sorbitan palmitate 20 EO or Polysorbate 40 sold under the name “Montanox 40 DF®” by the company SEPPIC, and sorbitan laurate 20 EO or polysorbate 20 sold under the name “Tween 20®” by the company ICI.

In this group, mention may also be made of alkyl or alkenyl esters of polyglycerol comprising at least 10 glycerol-based units, optionally oxyethylenated, such as, for example, polyglyceryl-10 laurate sold under the name “Decaglyn 1-L®” by the company Nikko Chemicals.

Mention may also be made of alkyl ethers, alkenyl ethers, alkyl esters and alkenyl esters of mono- or polysaccharides, such as, for example, those derived from compounds chosen from glucose, fructose, galactose, maltose and lactose, such as, for example, 1- and 6-monoesters of D-fructose, 1- and 6-monoesters of decylglucose and 1-and 6-monoesters of decylpolyglucose.

Non-limiting representative compounds of N-acylamino acids and derivatives thereof, and peptides N-acylated with at least one group chosen from alkyl and akenyl groups and salts thereof, may be compounds, wherein said alkyl and said alkenyl groups comprise at least 12 carbon atoms.

According to the invention, the term “amino acid” means α-, β- and γ-amino acids. As salts of N-acylamino acids, mention may be made, for example, of N-acyl glutamates chosen from monosodium cocoyl glutamate, monosodium lauroyl glutamate, and disodium C₁₄-C₂₀ alkyl glutamate, wherein the C₁₄-C₂₀ alkyl groups are derived from hydrogenated tallow, sold, respectively, under the names “Acylglutamate CS-11®”, “Acylglutamate LS-11®” and “Acylglutamate HS-21®” by the company Ajinomoto.

Mention may also be made of N-acyl lysines such as the lauroyl lysine sold under the name “Amihope LL®” by the company Ajinomoto.

Non-limiting examples of N-acylamino acid derivatives and salts thereof may be chosen from N-acyl sarcosinates such as sodium lauroyl sarcosinate, sold under the name “Oramix L30®” by the company SEPPIC, sodium myristoyl sarcosinate sold under the name “Nikkol Sarcosinate MN®” by the company Nikko Chemicals, and sodium palmitoyl sarcosinate sold under the name “Nikkol Sarcosinate PN®” by the company Nikko Chemicals.

Among the N-acyl peptides which may be used in accordance with the invention are those derived from all or a portion of collagen or keratin, such as the sodium lauroyl collagen and palmitoyl keratin sold under the names “Proteol B 30®” and “Lipacide PK®” by the company SEPPIC.

Non-limiting representatives of alkyl ethers, alkenyl ethers, alkyl ester sulfates and alkenyl ester sulfates, derivatives and salts thereof, are those chosen from alkyl and alkenyl groups comprising at least 12 carbon atoms.

Non-limiting examples of the alkyl ether sulfates and alkenyl ether sulfates used in accordance to the invention, are compounds chosen from alkyl ether sulfate salts, such as, for example, sodium lauryl ether sulfate.

Non-limiting examples of the alkyl ester sulfates and alkenyl ester sulfates used in accordance to the invention, may be chosen from isethionic acid esters and salts thereof, such as, for example, sodium cocoyl isethionate sold under the name “Geropon AC 78®” by the company Rhône-Poulenc.

Examples of polyoxyethylenated alkyl fatty esters, polyoxyethylenated alkenyl fatty esters, polyoxyethylenated alkyl fatty ethers, polyoxyethylenated alkenyl fatty ethers may be chosen from alkyl groups and alkenyl groups, wherein said alkyl groups and said alkenyl groups have at least 12 carbon atoms. In one embodiment of the invention, polyoxyethylenated fatty esters and ethers are chosen from compounds having at least 20 ethylene oxide units, such as, for example, PEG-20 stearate, laureth-23, oleth-20 and PEG-25 phytosterol.

Among the polyoxyethylenated alkyl or alkenyl carboxylic acids and salts thereof which may be used according to the invention are those comprising at least 10 ethylene oxide units, such as, for example, laureth-10 carboxylic acid and oleth-10 carboxylic acid.

Among the N-alkyl betaines and alkenyl betaines which may be used according to the invention, are those for which the alkyl groups and the alkenyl groups comprise at least 12 carbon atoms, such as, for example, lauryl amidopropyl betaine and oleyl amidopropyl betaine.

Among the alkyl trimethylammoniums, alkenyl trimethylammoniums and salts thereof which may be used according to the invention, are those for which the alkyl groups and the alkenyl groups comprise at least 12 carbon atoms. Non-limiting examples of salts which may be employed chosen from bromides and chlorides, such as, cocoyltrimethylammonium chloride and cetyltrimethylammonium bromide.

The binder phase may have a pH, for example, ranging from 5 to 8, such as, for example, from 6 to 7.

The binder phase according to the invention is stable when stored for 2 months at a temperature ranging from 4 to 45° C. without showing change in macroscopic appearance, microscopic appearance, color and odor.

The cubic gel particles of the invention may further comprise, or if there is a difference, be formed from at least one water-insoluble ionic amphiphilic lipid in an amount ranging, for example, from 0.0005% to 5% by weight relative to the total weight of the composition, such as, for example, from 0.001% to 2% by weight relative to the total weight of the composition.

Non-limiting examples of lipids which may be used in accordance with the invention are chosen from:

phospholipids chosen from natural phospholipids, such as, soya lecithin and egg lecithin, chemically modified phospholipids and enzymatically modified phospholipids, such as, hydrogenated lecithin and the sodium salt of phosphatidic acid, and synthetic phospholipids, such as, dipalmitoylphosphatidyl choline;

phosphoric esters of fatty alcohols, such as, monocetyl phosphate and sodium salts of monocetyl phosphate and potassium salts of monocetyl phosphate, sold under the name “Monafax 160®” by the company Mona, and dimyristyl phosphate, sodium salts of dimyristyl phosphate and potassium salts of dimyristyl phosphate, sold under the name “Mexoryl SY®” by the company Chimex;

N-acyl derivatives of glutamic acid, such as monosodium stearoyl glutamate sold under the name “Acylglutamate HS11®” by the company Ajinomoto, and the monosodium cocoyl-C₁₄-C₂₀ alkyl glutamate mixture, wherein said C₁₄-C₂₀ alkyl group is derived from hydrogenated tallow, sold under the name “Acylglutamate GS11®” by the company Ajinomoto;

sodium cetyl sulfate sold under the name “Nikkol SCS®” by the company Nikko Chemicals;

sodium cocoyl monoglyceride sulfate sold under the name “Nikkol SGC 80 N®” by the company Nikko Chemicals, and

quaternary ammonium derivatives chosen from behenyltrimethylammonium chloride, dilauryldimethylammonium chloride, distearyldimethylammonium chloride, 4,5-dihydro-1-methyl-2-(C₁₄-C₂₀)alkyl-1-(2-(C₁₄-C₂₀)alkylaminoethyl)imidazolium methyl sulfate, wherein the C₁₄-C₂₀ alkyl groups are derived from hydrogenated tallow, sold, for example, under the name of “Rewoquat W75H®” by the company Rewo Chemische, dialkylhydroxyethylmethylammonium methyl sulfate, wherein the alkyl groups are derived from optionally hydrogenated tallow, sold under the name “Stepanquat VP 85®” by the company Stepan and Quaternium-82 sold by the company SEPPIC under the name “Amonyl DM®”.

The incorporation of these water-insoluble ionic amphiphilic lipids may impart to the cubic gel particles a surface charge which results in an electrostatic repulsion between the particles.

The binder phase according to the invention, as defined above, may be obtained through fragmentation (with the aid of a homogenizer) of the at least one phytanetriol-based cubic gel, water, at least one water-soluble surface-active agent comprising optionally saturated, fatty chains, as defined above. Optionally, at least one compound chosen from water-insoluble ionic amphiphilic lipids and active principles chosen from hydrophilic active principles and lipophilic active principles may be incorporated into the binder phase.

The cubic gel particles may be obtained by various suitable mechanical means, for example, a high-shear homogenizer of a rotor-stator type such as a “Virtis” homogenizer, and a high-pressure homogenizer operating at a pressure ranging from 200 and 1 800 bar (20 to 180 MPa).

The average size of the particles in the dispersion according to the invention, as defined above, may, for one example, range from 0.05 to 1 μm, and another example, less than or equal to 0.5 μm. The particle size of the dispersion may also be modified by varying the nature and concentration of the water-soluble, fatty-chain surface-active agent used.

At this stage in the preparation, it is possible to incorporate into the cubic gel particles of the dispersions as defined above, at least one active principle, such as, for example, hydrophilic active principles and lipophilic active principles.

Needless to say, by virtue of the specific structure of the cubic gel particles, it is possible to incorporate therein both hydrophilic active principles and lipophilic active principles, even though these active principles have a certain level of incompatibility.

Non-limiting examples of various active principles may be chosen from:

antioxidants or free-radical scavengers chosen from proteins, enzymes, lactoperoxidase, lactoferrin, peptides and derivatives thereof, sequestrants, flavonoids, chlorophylline, ethoxyquine, guanosine, tocopherols and derivatives thereof, ascorbyl palmitate, β-carotene, vitamin E and derivatives thereof, vitamin C and derivatives thereof and vitamin A and derivatives thereof,

moisturizing and wetting agents chosen from hyaluronic acid, and its sodium salt, β-glycerophosphate, glycerol, sorbitol and panthenol,

UV screening agents chosen from the products sold under the names “Eusolex 232®” by the company Merck, “Parsol 1789®” and “Parsol MCX®” by the company Givaudan-Rouré, “Mexoryl SX®” by the company Chimex and “Uvinul T150®” by the company BASF,

keratolytic agents chosen from proteolytic enzymes, salicylic acid and derivatives thereof, such as 5-n-dodecanoylsalicylic acid, and retinoic acid and derivatives thereof,

tanning accelerators chosen from caffeine, and tyrosine derivatives, such as, glucose tyrosinate and the disodium salt of N-L-malyltyrosine,

depigmenting agents chosen from kojic acid, glycolic acid, vitamin C, magnesium ascorbyl phosphate, arbutin, and derivatives thereof,

natural colorants chosen from dyestuffs extracted from plants, for example, chlorophylline and β-carotene, and dyestuffs extracted from animals, for example, cochineal carmine, and caramel,

self-tanning agents chosen from dihydroxyacetone and indoles,

liporegulators chosen from β-orizanol, extract of Centella asiatica comprising genin and asiatic acid, caffeine and theophylline,

anti-ageing and anti-wrinkle agents chosen from hydroxy acids, for example, glycolic acid, n-octanoyl salicylic acid, retinol and derivatives thereof, such as, retinyl acetate, palmitate, propionate, and retinoids,

antiinflammatories and cicatrizing agents chosen from α-bisabolol, corticoids, 18-β-glycyrrhetinic acid and its salts such as, for example, its ammonium salt, extract of Centella asiatica, and aloe vera,

antibacterial and antifungal agents chosen from benzalkonium chloride, chlorhexidine, hexetidine and hexamidine,

insect repellents, chosen from diethyltoluamides and dimethyltoluamides,

deodorants chosen from hexachlorophene, and the product Triclosan sold under the name “Irgasan DP 300®” by the company Ciba Geigy,

antidandruff agents chosen from octopirox, and pyridinethione derivatives such as those sold under the name “Omadine®” by the company Olin,

agents for preventing hair loss, chosen from methylnicotinate, hexylnicotinate, and Minoxidil,

hair dyes chosen from oxidation bases, oxidation couplers, direct dyes and self-oxidizing dyes,

permanent-wave reducing agents chosen from thioglycolic acid, cysteine, cysteamine, n-acetylcysteine, N-acetylcysteamine and glyceryl thioglycolate, and

skin and hair conditioners chosen from cationic polymers and cations.

The binder phase according to the invention may thus comprise at least one type of particle chosen from hydrophilic active principles, and lipophilic active principles. Examples of hydrophilic and lipophilic active principles, include compounds such as, hydrophilic and lipophilic UV screening agents.

After formation of the cubic gel particles, said at least one dispersing and stabilizing agent is generally located on the outside of said particles.

In one embodiment of the invention, the binder phase further comprises an oily phase, wherein said oily phase optionally comprises at least one compound chosen from lipophilic additive and active principle. These compounds may be incorporated into the dispersion and mechanically stirred with the aid of, for example, a “Virtis®” type homogenizer.

It is thus possible to disperse the oily phase into the continuous aqueous phase in the form of droplets with an average size ranging, for example, from 0.1 micron to 10 microns. This oily phase comprises at least one oil chosen from plant origin, animal origin, mineral origin and synthetic origin.

Non-limiting representative oils which may be used in accordance with the invention, are chosen from mink oil, turtle oil, soybean oil, grape pip oil, sesame oil, corn oil, rapeseed oil, sunflower oil, cotton oil, avocado oil, olive oil, castor oil, jojoba oil, groundnut oil, hydrocarbon oils, for example, chosen from liquid paraffins, squalane and petroleum jelly, fatty esters, for example, chosen from isopropyl myristate, isopropyl palmitate, butyl stearate, hexyl laurate, isononyl isononanoate, 2-ethylhexyl palmitate, 2-hexyldecyl laurate, 2-octyldecyl palmitate, 2-octyldodecyl myristate, 2-octyldodecyl lactate, 2-diethylhexyl succinate, diisostearyl malate, glyceryl triisostearate, diglyceryl triisostearate, perfluoro oils, higher fatty acids, for example, chosen from myristic acid, palmitic acid, stearic acid, behenic acid, oleic acid, linoleic acid, linolenic acid and isostearic acid.

Non-limiting representatives of synthetic oils which may be used in accordance to the invention may be chosen from hydrocarbons, such as, for example, isohexadecane, polydecene and polyisobutene, fatty alcohols, such as, for example, octyldodecanol, cetanol, stearyl alcohol and oleyl alcohol, esters, such as, for example, glycerides of essential fatty acids, triglycerides of capric acid, triglycerides of acrylic acid, and mixtures thereof, esters of fatty alcohols, esters of linear and branched fatty acids, such as, for example, purcellin oil (stearyl octanoate).

Additional examples of synthetic oils which may also be used in the compositions according to the invention, are chosen from linear type silicone oils, such as, for example, polydimethylsiloxane, cyclic type silicone oils, such as, for example, cyclopentadimethylsiloxane and organomodified type silicone oils, such as, for example, oxyethylenated oxypropylenated polyphenyltrimethylsiloxane and polydimethylsiloxane.

The above mentioned oils may be used alone or as mixtures.

It is also possible to incorporate into the continuous aqueous phase of the dispersion of the present invention at least one cosmetically or dermatologically acceptable compound, for example, chosen from hydrophilic active principles, such as, conventional moisturizers and additives.

In one embodiment of the invention, said at least one cosmetically or dermatologically acceptable compound may be chosen from preserving agents, fragrances, dyestuffs, and gelling agents, etc.

Among the gelling agents which may be used in the compositions according to the invention, mention may be made of, for example, gelling agents chosen from cellulose derivatives, such as, for example, hydroxyethylcellulose and alkyhydroxyethylcelluloses, algal derivatives, such as, for example, satia gum, natural gums, such as, tragacanth, synthetic polymers, such as, mixtures of polycarboxyvinylic acids, such as, for example, those sold under the names “Carbopol” by the company Goodrich and “Synthalen” by the company 3V SA.

The gelling agent may be present in the composition according to the present invention in an amount, for example, ranging from 0.1% to 2% by weight relative to the total weight of the binder phase.

In one embodiment of the invention, the composition comprises at least one gelling agent.

In addition to the dispersed cubic gel particles, the compositions according to the invention may further comprise liposomes optionally comprising active principles.

The presence of phytanetriol in the compositions according to the invention may impart good moisturizing power to the composition.

The binder phase may further comprise at least one fatty substance other than those already mentioned, chosen from waxes and pasty fatty substances.

The pasty fatty compounds may be defined with the aid of at least one physicochemical property chosen from:

a viscosity ranging from 0.1 to 40 Pa.s (1 to 400 poises), such as, for example, from 0.5 to 25 Pa.s, measured at 40° C. with a Contraves TV rotary viscometer equipped with an MS-r3 or MS-r4 spindle, at a frequency of 240 rpm, and

a melting point ranging from 25 to 70° C., such as, for example, from 25 to 55° C.

Waxes which may be used in the compositions of the invention, may, for example, be chosen from beeswaxes, lanolin waxes, Chinese insect waxes, carnauba wax, candelilla wax, ouricury wax, cork fiber waxes, sugar cane waxes, Japan waxes, hydrogenated jojoba waxes, hydrogenated oils, such as, for example, hydrogenated sunflower oil, hydrogenated castor oil, hydrogenated coconut oil and hydrogenated lanolin, paraffins, microcrystalline waxes, Montan waxes, ozokerites, polyethylene waxes, waxes obtained by Fischer-Tropsch synthesis, waxy copolymers and waxy esters thereof, and silicone waxes, such as, for example, polyalkoxysiloxanes, polyalkylsiloxanes, and mixtures thereof.

The binder phase may optionally further comprise a volatile portion such as, for example, at least one volatile oil.

The term “volatile oil” means any compound capable of evaporating upon contact with the skin. Oils whose flashpoint is high enough to allow these oils to be used in formulation, and low enough to obtain the desired evanescent effect may be chosen. In one embodiment of the invention, oils with flashpoints ranging from 40 to 100° C. are chosen.

Non-limiting examples of these volatile compounds may be chosen from hydrocarbon-based oils, such as, for example, isoparaffins such as, isododecane, cyclic volatile silicones comprising from 3 to 8 silicon atoms, such as, 4 to 6 silicon atoms, for example, chosen from cyclotetradimethylsiloxane, cyclopentadimethylsiloxane and cyclohexadimethylsiloxane, such as, the products sold under the names “DC Fluid 244”, “DC Fluid 245”, “DC Fluid 344” and “DC Fluid 345” by the company Dow Corning, linear volatile silicones comprising from 2 to 9 silicon atoms, for example hexamethyldisiloxane, hexylheptamethyltrisiloxane and octylheptamethyltrisiloxane.

The binder phase may further comprise at least one additive chosen from lipophilic cosmetic active agents and liposoluble ingredients generally used in cosmetics, such as, for example, fragrances and sunscreens. These additives may be present in an amount ranging from 1% to 70% by weight relative to the total weight of the binder phase.

The compositions according to the invention may further comprise silicone resins comprising at least one unit chosen from R₃SiO_{1/2}, R₂SiO_{2/2}, RSiO_{3/2} and SiO_{4/2}, wherein, R is chosen from a hydrogen atom, C₁-C₆ alkyl groups and a phenyl groups.

Needless to say, a person skilled in the art will take care to select the type and concentration of any of these optionally additional compounds such that the advantageous properties of the composition according to the invention are not substantially adversely affected by the addition envisaged.

When the compositions of the invention are face or body powders, they may comprise, for example, at least 70% by weight of the pulverulent phase relative to the total weight of the composition, such as, for further example, from 70% to 99.9% by weight of the pulverulent phase relative to the total weight of the composition.

The binder phase may be present in the composition according to the invention in an amount less than or equal to 30% by weight relative to the total weight of the composition, such as, for example, from 0.1% to 23% by weight and further still from 3% to 20% by weight, relative to the total weight of the composition.

The composition according to the invention may be in the form of a powder, chosen from, for example, a compact powder, a free powder, a pressed powder and a cast powder. In the case of a free powder, for example, the binder phase may be present in the composition according to the invention, in an amount, for example, less than or equal to 15% by weight relative to the total weight of the composition, such as, for example, from 1% to 5% by weight relative to the total weight of the composition. For a compact powder, the content of binder phase may be present in an amount, for example, ranging from 1 to 30% by weight relative to the total weight of the composition, such as, for example, from 1% to 20% by weight relative to the total weight of the composition.

When the compositions according to the invention are in the forms chosen from foundations, lipsticks, mascaras, eyeliners and make-up products for the body, the binder phase may be present in an amount ranging from 50% to 99.9% by weight relative to the weight of the final composition. In one embodiment of the invention, these compositions may be in a form chosen from fluid products, cast products, creams, and sticks, etc.

In another embodiment of the invention, the compositions may also comprise a binder phase present in an amount ranging from 30% to 50% by weight relative to the total weight of the composition. The final product may act both as a powder and as a foundation. Such a product is practical and advantageous since it is compact and easy to transport. Furthermore, such a product may combine both the cosmetic qualities of a powder, such as staying power and powdery effect on the skin, with those of a foundation, such as comfortable feel, moisturization, slipperiness and the ease of application. Such a product may possess at least one of the following properties: creaminess, pleasant sensations of freshness and no sensation of dryness and tautness. The products may also be used as a make-up product for the body.

The compositions according to the invention may be prepared according to known methods for preparing cosmetic sticks, cast products, emulsions and powders.

In one embodiment of the invention, foundations, lipsticks, mascaras, eyeliners and make-up products for the body may be prepared, as desired, by use of a kit comprising a plurality of separate containers. At least one container comprises the binder phase prepared beforehand. At least one other container comprises pigments of different colors, for example, those chosen from, active agents in pulverulent form, fillers, nacres and flakes and mixtures thereof.

The invention also relates to a method for preparing the make-up product by placing a few grams of binder phase into the palm of the hand, and then adding at least one pulverulent compound to the binding phase and mixing said at least one pulverulent and the binder phase together using a finger. After swirling a few times, such as, for example, 20 times, a homogeneous composition can be obtained, in which the pulverulent compounds are well dispersed, and are easy to apply to keratinous materials, such as skin and mucous membranes. The make-up obtained can be homogeneous, uniform and comfortable.

Thus, the present invention also relates to a process for making up keratin materials of human beings, comprising:

a) mixing at least one pulverulent compound with an effective amount of a binder phase comprising a dispersion of cubic gel particles to form a homogeneous composition; and

b) applying said composition to said keratinous materials.

The mixing may be carried out, for example, in the palm of the hand or in any container of any volume. The term “effective amount” means the amount of binder phase which is required to disperse the pulverulent compounds and obtain a homogeneous composition. A person skilled in the art is capable of determining this amount as a function of the amount of pulverulent compounds used. The expression “homogeneous composition” means a composition in which pulverulent compounds are finely and uniformly dispersed, wherein the final composition forms virtually no aggregates or lumps. For example, in one embodiment of the invention, the relationship between the at least one pulverulent and the binder phase can be defined as a ratio, A/B, ranging from 100:1 to 1:1, wherein A is the mass in grams of the binder phase used and B is the mass in grams of pulverulent compounds used.

Additionally, mixing of the dispersion of cubic gel particles and of the pulverulent compounds may be carried out using any mixing device, such as, for example, a “Moritz” mixer (microdispersing turbomixer, at maximum speed for 10 minutes), an “ultra-turrax” mixer (with or without scraper paddles), and simply with a finger.

In one embodiment of the invention, combining at least one pulverulent with an effective amount of binder phase is carried out with a finger in the palm of the hand. It is possible, for example, to place about 0.2 g to 1 g (gram) of binder phase in the hand and to add about 0.01 g to 1 g of at least one pulverulent. This combination is then mixed with a finger until a homogeneous composition is obtained. Depending on what type of cosmetic care foundation is desired, titanium oxides, iron oxides, and alternatively cosmetic care active agents in pulverulent form may be mixed with the binder phase to obtain variously colored compositions. For example, red pigments such as certain iron oxides may be incorporated into the composition to obtain a lipstick.

Needless to say, the foundations and other make-up products according to the invention may also be prepared by conventional preparation methods, for example, by first pre-dispersing the pigments in a suitable fatty substance by grinding and stirring using, for example, a homogenizer such as a “Virtis®” machine.

Unless otherwise indicated, all numbers expressing quantities of ingredients, properties such as molecular weight, reaction conditions, and so forth used in the specification and claims are to be understood as being modified in all instances by the term “about”. Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

The examples which follow are intended to illustrate the invention without, however, limiting its scope. The invention is illustrated in greater detail in the examples which follow.

In the examples which follow, the amounts are given as percentages by weight relative to the total weight of the composition.

### EXAMPLE 1

The following compositions were prepared in the form of powders (the amounts are given as percentages by weight relative to the total weight of the composition):

| | |
|---|---|
| Phase A (Pulverulent Compounds): | |
| talc | X% |
| iron oxides | 2.74% |
| Nylon powder | 10% |
| titanium oxide | 1% |
| preserving agent | 0.2% |
| Phase B (Binder Phase): | |
| (dispersion of cubic gel particles) | Y% |
| with: | |

| | X% | Y% |
|---|---|---|
| Composition 1 | 77.06 | 9 |
| Composition 2 | 74.06 | 12 |
| Composition 3 | 71.06 | 15 |

The composition of the binder phase (dispersion of cubic gel particles) was as follows (the amounts are given as percentages by weight relative to the total weight of the binder phase):

| | |
|---|---|
| isopropyl myristate | 1.64% |
| castor oil | 2.46% |
| liquid petroleum jelly | 12.36% |
| liquid lanolin | 1.26% |
| water | 70.95% |
| imidazolinylurea | 0.3% |
| glycerol | 5% |
| monosodium salt of N-stearoyl-L-glutamic | 0.03% |
| acid sold under the name “Acylglutamate | |
| HS-11” by the company Ajinomoto | |
| phytanetriol | 2.97% |
| petroleum jelly | 2.28% |
| chlorphenesine | 0.25% |
| oxyethylenated sorbitan monopalmitate | 0.5% |
| (40 EO) | |

This composition was prepared in the following way: an aqueous dispersion of cubic gel particles was obtained by mixing together 2.97 g of phytanetriol and 0.03 g of monosodium salt of N-stearoyl-L-glutamic acid and 1.28 g of water, to which was added 75.17 g of an aqueous solution comprising 0.5 g of oxyethylenated sorbitan monopalmitate and 5 g of glycerol. The mixture was then predispersed, after which it was homogenized at room temperature, using a “Virtis®” homogenizer at 35 000 rpm for 5 minutes, this stirring being repeated 4 times.

The preserving agents (chlorphenesine and imidazolinylurea) and the fatty substances (isopropyl myristate, castor oil, liquid petroleum jelly, liquid lanolin and petroleum jelly) were then added to the aqueous dispersion of cubic gel particles obtained. After stirring at room temperature using a Virtis® homogenizer at 35 000 rpm for 5 minutes, this stirring being repeated 5 times, a stable, homogeneous dispersion was obtained.

For each composition 1, 2, 3, a mixture of the compounds of phase A (pulverulent compounds) was first prepared in a Baker-Perkins turbomixer. Phase B (binder phase) was then added thereto with stirring. The two phases were mixed together until a homogeneous product was obtained. The temperature of the mixture was kept at 35° C.

Powders which show good cohesion of the pulverulent compounds were obtained. These compositions afforded a sensation of freshness when applied to the skin.

### EXAMPLE 2

A dispersion of cubic gel particles was prepared as follows:

| | |
|---|---|
| hydrogenated isoparaffin | 7.8% |
| isostearyl neopentanoate | 2.6% |
| preserving agents | 0.61% |
| fragrance | 0.1% |
| pentasodium salt of ethylenediaminetetra- | 0.1% |
| methylenephosphonic acid at 33% in water | |
| alkyl hydroxyethylcellulose | 1% |
| cyclohexadimethylsiloxane | 11.6% |
| phytanetriol | 2.97% |
| demineralized water | 69.19% |
| glycerol | 3% |
| monosodium salt of N-stearoyl-L-glutamic | 0.03% |
| acid sold under the name “Acylglutamate | |
| HS-11” by the company Ajinomoto | |
| oxyethylenated sorbitan monopalmitate | 1% |
| (40 EO) | |

An aqueous dispersion of cubic gel particles was obtained by mixing together 2.97 g of phytanetriol and 0.03 g of monosodium salt of N-stearoyl-L-glutamic acid and 1.28 g of water, to which 57.91 g of an aqueous solution comprising 1 g of oxyethylenated sorbitan monopalmitate and 3 g of glycerol was added. The mixture was then predispersed, after which it was homogenized at room temperature, using a Virtis® homogenizer at 35 000 rpm for 5 minutes, the stirring being repeated 4 times.

The fragrance and the fatty substances (hydrogenated isoparaffin, isostearyl neopentanoate and cyclohexadimethylsiloxane) were then added to the aqueous dispersion of cubic gel particles obtained. The mixture was then homogenized at room temperature using a Soavi® high-pressure homogenizer, by 4 homogenization treatments at 600 bar.

A solution obtained by mixing together alkyl hydroxyethylcellulose and preserving agents with 14 g of demineralized water was then added thereto. The mixture was homogenized at room temperature using a Heidolph RZR 50® paddle stirrer at 50 rpm for 30 minutes.

A stable and homogeneous dispersion of cubic gel particles was thus obtained.

0.36 g of this dispersion was placed in the palm of the hand and 0.03 g of pure pigment (yellow iron oxide) was added thereto. These materials were mixed together with the index finger by a circular motion (about 20 swirls). A foundation in the form of a fluid cream in which the pigments were very well dispersed was obtained.

This cream was then applied directly to the face and spread on, to give a homogeneous and uniform make-up effect.

### EXAMPLE 3

The dispersion of cubic gel particles of Example 2 was stored in a bottle. Yellow iron oxides, black iron oxides, red iron oxides, white titanium oxides, nacres and flakes were stored in separate containers. Active agents in pulverulent form and fillers were stored in other separate containers. A make-up kit for preparing one's own foundation at home was thus obtained by simply mixing together in the palm of the hand, a dispersion of cubic gel particles and at least one pulverulent compound chosen from pigments, fillers, nacres, flakes and active agents.

### EXAMPLE 4

The following composition (the amounts are given as percentages by weight relative to the total weight of the composition) was prepared:

| | |
|---|---|
| talc | 46.06% |
| iron oxides | 2.74% |
| Nylon powder | 10% |
| titanium oxide | 1% |
| preserving agent | 0.2% |
| dispersion of cubic gel particles | 40% |

The dispersion of cubic gel particles used was the same as that in Example 1.

A product whose texture was more compact than a cream but which was not a powder was obtained. This product may be used both as a foundation and as a face powder. It produced a powder-like effect on the skin while at the same time giving a sensation of freshness when applied. It was comfortable and easy to apply.

What is claimed is:

1. A make-up composition comprising a pulverulent phase and a binder phase, wherein said binder phase comprises a dispersion of cubic gel particles.

2. A make-up composition according to claim 1, wherein said pulverulent phase comprises at least one pulverulent chosen from pigments, nacres, fillers, and flakes.

3. A make-up composition according to claim 2, wherein said pigments are chosen from titanium dioxide, zirconium dioxide, cerium dioxide, zinc oxide, iron oxide, chromium oxide, nanotitaniums, nanozincs, ferric blue, carbon black, lakes, and acidic dyes.

4. A make-up composition according to claim 2, wherein said pigments are coated with a material chosen from silicone compounds and polymers.

5. A make-up composition according to claim 4, wherein said silicone compounds are chosen from polydimethylsiloxanes and said polymers are chosen from polyethylenes.

6. A make-up composition according to claim 3, wherein said lakes are chosen from calcium salts, barium salts, aluminium salts, and zirconium salts.

7. A make-up composition according to claim 3, wherein said acidic dyes are chosen from halo acid dyes, azo dyes and anthraquinone dyes.

8. A make-up composition according to claim 2, wherein said pigments are present in an amount ranging from 0.05% to 80% by weight relative to the total weight of said composition.

9. A make-up composition according to claim 8, wherein said pigments are present in an amount ranging from 0.5% to 50% by weight relative to the total weight of said composition.

10. A make-up composition according to claim 2, wherein said fillers are chosen from talc, mica, silica, kaolin, Nylon powder, poly-β-alanine powder, polyethylene powder, Teflon, lauroyllysine, starch, boron nitride, bismuth oxychloride, tetrafluoroethylene polymer powders, polymethyl methacrylate powders, polyurethane powders, polystyrene powders, polyester powders, synthetic hollow microspheres, microsponges, silicone resin microbeads, zinc oxide, titanium oxide, zirconium oxide, cerium oxide, precipitated calcium carbonate, magnesium carbonate, magnesium hydrocarbonate, hydroxyapatite, hollow silica microspheres, glass microcapsules, ceramic microcapsules, and metal soaps derived from organic carboxylic acids comprising from 8 to 22 carbon atoms.

11. A make-up composition according to claim 10, wherein said metal soaps are derived from organic carboxylic acids comprising from 12 to 18 carbon atoms.

12. A make-up composition according to claim 10, wherein said metal soaps are chosen from zinc stearate, magnesium stearate, lithium stearate, zinc laurate and magnesium myristate.

13. A make-up composition according to claim 2, wherein said fillers are present in an amount ranging from 0.05% to 99% by weight relative to the total weight of the composition.

14. A make-up composition according to claim 2, wherein said nacres are chosen from natural mother-of-pearl, mica coated with titanium oxide, mica coated with iron oxide, mica coated with natural pigment, mica coated with bismuth oxychloride, and colored titanium mica.

15. A make-up composition according to claim 2, wherein said nacres are present in an amount ranging from 0.05% to 80% by weight relative to the total weight of the composition.

16. A make-up composition according to claim 15, wherein said nacres are present in an amount ranging from 2% to 50% by weight relative to the total weight of the composition.

17. A make-up composition according to claim 2, wherein said at least one pulverulent is chosen from flakes.

18. A make-up composition according to claim 1, wherein said pulverulent phase is present in an amount of at least 0.1% by weight relative to the total weight of the composition.

19. A make-up composition according to claim 18, wherein said pulverulent phase is present in an amount of at least 2% by weight relative to the total weight of the composition.

20. A make-up composition according to claim 1, wherein said binder phase is an aqueous continuous phase comprising:
(i) phytanetriol, wherein said phytanetriol is present in an amount ranging from 0.1% to 15% by weight relative to the total weight of the binder phase, and
(ii) at least one dispersing and stabilizing agent chosen from surface-active agents chosen from optionally saturated, linear and branched surface-active agents comprising fatty chains having from 8 to 22 carbon atoms, wherein said at least one dispersing and stabilizing agent is present in an amount ranging from 0.1% to 3% by weight relative to the total weight of said binder phase and further wherein said surface-active agents are water-soluble at room temperature.

21. A make-up composition according to claim 20, wherein said phytanetriol is present in an amount ranging from 0.5% to 10% by weight relative to the total weight of the binder phase.

22. A make-up composition according to claim 21, wherein the weight ratio of said phytanetriol to said at least one dispersing and stabilizing agent ranges from 2:1 to 200:1.

23. A make-up composition according to claim 1, wherein said binder phase is chosen from compositions comprising:
(1) an aqueous phase, wherein said aqueous phase is present in an amount ranging from 60% to 98% by weight relative to the total weight of said composition; and
(2) an oily phase, wherein said oily phase is present in an amount ranging from 2% to 40% by weight relative to the total weight of said composition, and further wherein said oily phase is dispersed in said aqueous phase and is stabilized with the aid of cubic gel particles, said cubic gel particles comprise:
(i) at least one compound chosen from phytanetriol, N-2-alkoxycarbonyl derivatives of N-methylglucamine, and unsaturated fatty acid monoglycerides, wherein said at least one compound is present in an amount ranging from 0.1% to 15% by weight relative to the total weight of the composition; and
(ii) at least one dispersing and stabilizing agent chosen from surface-active agents chosen from optionally saturated, linear and branched surface-active agents comprising fatty chains having 8 to 22 carbon atoms, wherein said at least one dispersing and stabilizing agent is present in an amount ranging from 0.05% to 3% by weight relative to the total weight of said composition and further wherein said surface-active agents are water-soluble at room temperature.

24. A make-up composition according to claim 23, wherein the weight ratio of said cubic gel particles to said oily phase ranges from 0.02:1 to 1:1.

25. A make-up composition according to claim 24, wherein the weight ratio of said cubic gel particles to said oily phase ranges from 0.05:1 to 0.5:1.

26. A make-up composition according to claim 23, wherein the weight ratio of said cubic gel particles to said at least one dispersing and stabilizing agent ranges from 2:1 to 200:1.

27. A make-up composition according to claim 26, wherein the weight ratio of said cubic gel particles to said at least one dispersing and stabilizing agent is less than or equal to 50:1.

28. A make-up composition according to claim 20, wherein said at least one dispersing and stabilizing agent is chosen from:
polyol alkyl ethers, polyol alkenyl ethers, polyol alkyl esters, and polyol alkenyl esters;
N-acylated amino acids and derivatives thereof;
peptides, wherein said peptides are N-acylated with at least one group chosen from alkyl groups and alkenyl groups, and salts thereof;
alkyl ethers, alkenyl ethers, alkyl ester sulfates and alkenyl ester sulfates, derivatives and salts thereof;
polyoxyethylenated alkyl fatty ethers, polyoxyethylenated alkenyl fatty ethers, polyoxyethylenated alkyl fatty esters, and polyoxyethylenated alkenyl fatty esters;
polyoxyethylenated alkyl carboxylic acids, polyoxyethylenated alkenyl carboxylic acids and salts thereof;
N-alkyl betaines, and N-alkenyl betaines; and
alkyltrimethylammonium, alkenyltrimethylammonium and salts thereof.

29. A make-up composition according to claim 23, wherein said at least one dispersing and stabilizing agent is chosen from:
polyol alkyl ethers, polyol alkenyl ethers, polyol alkyl esters, and polyol alkenyl esters;
N-acylated amino acids and derivatives thereof;
peptides, wherein said peptides are N-acylated with at least one group chosen from alkyl groups and alkenyl groups, and salts thereof;
alkyl ethers, alkenyl ethers, alkyl ester sulfates and alkenyl ester sulfates, derivatives and salts thereof;
polyoxyethylenated alkyl fatty ethers, polyoxyethylenated alkenyl fatty ethers, polyoxyethylenated alkyl fatty esters, and polyoxyethylenated alkenyl fatty esters;
polyoxyethylenated alkyl carboxylic acids, polyoxyethylenated alkenyl carboxylic acids and salts thereof;
N-alkyl betaines, and N-alkenyl betaines; and
alkyltrimethylammonium, alkenyltrimethylammonium and salts thereof.

30. A make-up composition according to claim 23, wherein said N-2-alkoxycarbonyl derivatives of N-methylglucamine are chosen from at least one compound of formula (I): 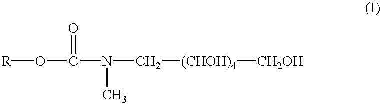
wherein R is chosen from branched (C₆-C₁₈) alkyl groups.

31. A make-up composition comprising a pulverulent phase and a binder phase, wherein said binder phase is chosen from compositions comprising:
(1) an aqueous phase, wherein said aqueous phase is present in an amount ranging from 60% to 98% by weight relative to the total weight of said composition; and
(2) an oily phase, wherein said oily phase is present in an amount ranging from 2% to 40% by weight relative to the total weight of said composition, and further wherein said oily phase is dispersed in said aqueous phase and is stabilized with the aid of cubic gel particles, said cubic gel particles comprise:
(i) a mixture comprising:
(a) phytanetriol in an amount ranging from 1% to 40% by weight relative to the total weight of said mixture; and
(b) at least one compound of formula (I): 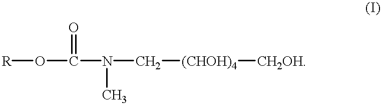
wherein R is chosen from branched (C₆-C₁₈) alkyl groups and said at least one compound of formula (I) is present in an amount ranging from 60% to 99% by weight relative to the total weight of said mixture; and
(ii) at least one dispersing and stabilizing agent chosen from surface-active agents chosen from optionally saturated, linear and branched surface-active agents comprising fatty chains having 8 to 22 carbon atoms, wherein said at least one dispersing and stabilizing agent is present in an amount ranging from 0.05% to 3% by weight relative to the total weight of said composition and further wherein said surface-active agents are water-soluble at room temperature.

32. A make-up composition according to claim 31, wherein said mixture comprises:
(a) phytanetriol in an amount ranging from 10% to 30% by weight relative to the total weight of said mixture; and
(b) at least one compound of formula (I) in an amount ranging from 70% to 90% by weight relative to the total weight of said mixture.

33. A make-up composition comprising a pulverulent phase and a binder phase, wherein said binder phase is chosen from compositions comprising:
(1) an aqueous phase, wherein said aqueous phase is present in an amount ranging from 60% to 98% by weight relative to the total weight of said composition; and
(2) an oily phase, wherein said oily phase is present in an amount ranging from 2% to 40% by weight relative to the total weight of said composition, and further wherein said oily phase is dispersed in said aqueous phase and is stabilized with the aid of cubic gel particles, said cubic gel particles comprise:
(i) a mixture comprising:
(a) phytanetriol in an amount ranging from 1% to 50% by weight relative to the total weight of said mixture; and
(b) at least one unsaturated fatty acid monoglyceride present in an amount ranging from 50% to 99% by weight relative to the total weight of said mixture; and
(ii) at least one dispersing and stabilizing agent chosen from surface-active agents chosen from optionally saturated, linear and branched surface-active agents comprising fatty chains having 8 to 22 carbon atoms, wherein said at least one dispersing and stabilizing agent is present in an amount ranging from 0.05% to 3% by weight relative to the total weight of said composition and further wherein said surface-active agents are water-soluble at room temperature.

34. A make-up composition according to claim 33, wherein said mixture comprises:
(a) phytanetriol in an amount ranging from 10% to 30% by weight relative to the total weight of said mixture; and
(b) at least one unsaturated fatty acid monoglyceride in an amount ranging from 70% to 90% by weight relative to the total weight of said mixture.

35. A make-up composition according to claim 1, wherein said cubic gel particles further comprise at least one water-insoluble ionic amphiphilic lipid.

36. A make-up composition according to claim 35, wherein said at least one water-insoluble ionic amphiphilic lipid is chosen from:
phospholipids chosen from soya lecithin, egg lecithin, hydrogenated lecithin, the sodium salt of phosphatidic acid, and dipalmitoylphosphatidyl choline;
phosphoric esters of fatty alcohols chosen from monocetyl phosphate, sodium salts of monocetyl phosphate, potassium salts of monocetyl phosphate, dimyristyl phosphate, sodium salts of dimyristyl phosphate, potassium salts of dimyristyl phosphate;
N-acyl derivatives of glutamic acid, chosen from monosodium stearoyl glutamate and the monosodium cocoyl-C₁₄-C₂₀ alkyl glutamate mixture, wherein said C₁₄-C₂₀ alkyl group is derived from hydrogenated tallow;
sodium cetyl sulfate;
sodium cocoyl monoglyceride sulfate; and
quaternary ammonium derivatives chosen from behenyltrimethylammonium chloride, dilauryldimethylammonium chloride, distearyldimethylammonium chloride, 4,5-dihydro-1-methyl-2-(C₁₄-C₂₀)alkyl-1-(2-(C₁₄-C₂₀)alkylaminoethyl)imidazolium methyl sulfate, wherein the C₁₄-C₂₀ alkyl groups are derived from hydrogenated tallow, and dialkylhydroxyethylmethylammonium methyl sulfate, wherein the alkyl groups are derived from optionally hydrogenated tallow.

37. A make-up composition according to claim 35, wherein said at least one water-insoluble ionic amphiphilic lipid is present in an amount ranging from 0.0005% to 5% by weight relative to the total weight of said composition.

38. A make-up composition according to claim 36, wherein said at least one water-insoluble ionic amphiphilic lipid is present in an amount ranging from 0.001% to 2% by weight relative to the total weight of said composition.

39. A make-up composition according to claim 1, wherein said cubic gel particles further comprise at least one active principle chosen from hydrophilic active principles and lipophilic active principles.

40. A make-up composition according to claim 39, wherein said at least one active principle is chosen from:
antioxidants and free-radical scavengers chosen from proteins, enzymes, lactoperoxidase, lactoferrin, peptides and derivatives thereof, sequestrants, flavonoids, chlorophylline, ethoxyquine, guanosine, tocopherols and derivatives thereof, ascorbyl palmitate, β-carotene, vitamin E and derivatives thereof, vitamin C and derivatives thereof and vitamin A and derivatives thereof;
moisturizing and wetting agents chosen from hyaluronic acid, sodium salts of hyaluronic acid, β-glycerophosphate, glycerol, sorbitol and panthenol;
UV screening agents;
keratolytic agents chosen from proteolytic enzymes, salicylic acid and derivatives thereof;
tanning accelerators chosen from caffeine, and tyrosine derivatives;
depigmenting agents chosen from kojic acid, glycolic acid, vitamin C, magnesium ascorbyl phosphate, arbutin, and derivatives thereof,
natural colorants chosen from dyestuffs extracted from plants, and dyestuffs extracted from animals;
self-tanning agents chosen from dihydroxyacetone and indoles,
liporegulators chosen from y-orizanol, extract of Centella asiatica comprising genin and asiatic acid, caffeine and theophylline,
anti-ageing and anti-wrinkle agents chosen from hydroxy acids, and derivatives thereof;
antiinflammatories and cicatrizing agents chosen from α-bisabolol, corticoids, 18-β-glycyrrhetinic acid, salts of 18-β-glycyrrhetinic acid, extract of Centella asiatica, and aloe vera;
antibacterial and antifungal agents chosen from benzalkonium chloride, chlorhexidine, hexetidine and hexamidine;
insect repellents chosen from diethyltoluamides and dimethyltoluamides;
deodorants chosen from hexachlorophene;
antidandruff agents chosen from octopirox, and pyridinethione derivatives;
agents for preventing hair loss, chosen from methylnicotinate, hexylnicotinate, and Minoxidil,
hair dyes chosen from oxidation bases, oxidation couplers, direct dyes and self-oxidizing dyes,
permanent-wave reducing agents chosen from thioglycolic acid, cysteine, cysteamine, n-acetylcysteine, N-acetylcysteamine and glyceryl thioglycolate, and
skin and hair conditioners chosen from cationic polymers and cations.

41. A make-up composition according to claim 23, wherein said oily phase comprises at least one oil chosen from plant origin, animal origin, mineral origin and synthetic origin.

42. A make-up composition according to claim 23, wherein said oily phase comprises at least one oil chosen from mink oil, turtle oil, soybean oil, grape pip oil, sesame oil, corn oil, rapeseed oil, sunflower oil, cotton oil, avocado oil, olive oil, castor oil, jojoba oil, groundnut oil, hydrocarbon oils chosen from liquid paraffins, squalane and petroleum jelly, fatty esters chosen from isopropyl myristate, isopropyl palmitate, butyl stearate, hexyl laurate, isononyl isononanoate, 2-ethylhexyl palmitate, 2-hexyldecyl laurate, 2-octyldecyl palmitate, 2-octyldodecyl myristate, 2-octyldodecyl lactate, 2-diethylhexyl succinate, diisostearyl malate, glyceryl triisostearate, and diglyceryl triisostearate, perfluoro oils, higher fatty acids chosen from myristic acid, palmitic acid, stearic acid, behenic acid, oleic acid, linoleic acid, linolenic acid and isostearic acid, hydrocarbons chosen from isohexadecane, polydecene and polyisobutene, fatty alcohols chosen from octyldodecanol, cetanol, stearyl alcohol and oleyl alcohol, esters chosen from glycerides of essential fatty acids, triglycerides of capric acid, and triglycerides of acrylic acid, esters of fatty alcohols, esters of linear and branched fatty acids chosen from purcellin oil, linear type silicone oils chosen from polydimethylsiloxane, cyclic type silicone oils chosen from cyclopentadimethylsiloxane and organomodified type silicone oils chosen from oxyethylenated oxypropylenated polyphenyltrimethylsiloxane and polydimethylsiloxane.

43. A make-up composition according to claim 1, further comprising at least one gelling agent.

44. A make-up composition according to claim 43, wherein said at least one gelling agent is chosen from cellulose derivatives chosen from hydroxyethylcellulose and alkyhydroxyethylcelluloses, algal derivatives chosen from satia gum, and natural gums, synthetic polymers chosen from mixtures of polycarboxyvinylic acids.

45. A make-up composition according to claim 43, wherein said at least one gelling agent is present in an amount ranging from 0.1% to 2% by weight relative to the total weight of the binder phase.

46. A make-up composition according to claim 1, wherein said binder phase further comprises at least one fatty substance chosen from waxes and pasty fatty substances.

47. A make-up composition according to claim 46, wherein said waxes are chosen from beeswaxes, lanolin waxes, Chinese insect waxes, carnauba wax, candelilla wax, ouricury wax, cork fiber waxes, sugar cane waxes, Japan waxes, hydrogenated jojoba waxes, hydrogenated oils, paraffins, microcrystalline waxes, Montan waxes, ozokerites, polyethylene waxes, waxes obtained by Fischer-Tropsch synthesis, waxy copolymers and waxy esters thereof, and silicone waxes.

48. A make-up composition according to claim 1, wherein said binder phase further comprises at least one volatile oil.

49. An eye shadow, a face and body powder, a concealer product, a foundation, a make-up product for the body, a lipstick, a mascara, an eyeliner an anti-sun product or a self-tanning product comprising a pulverulent phase and a binder phase, wherein said binder phase comprises a dispersion of cubic gel particles.

50. A face or body powder comprising a pulverulent phase and a binder phase, wherein said binder phase comprises a dispersion of cubic gel particles and further wherein said binder phase is present in an amount less than or equal to 30% by weight relative to the total weight of the composition.

51. A face or body powder according to claim 50, wherein said binder phase is present in an amount ranging from 0. 1% to 23% by weight relative to the total weight of the composition.

52. A foundation, a lipstick, a mascara, an eyeliner or a make-up product for the body comprising a pulverulent phase and a binder phase, wherein said binder phase comprises a dispersion of cubic gel particles and further wherein said binder phase is present in amount ranging from 50% to 99.9% by weight relative to the weight of said composition.

53. A make-up composition according to claim 1, wherein said binder phase is present in an amount ranging from 30% and 50% by weight relative to the total weight of said composition.

54. A process for making-up or caring for keratinous materials comprising:
combining a pulverulent phase and a binder phase, wherein said binder phase comprises a dispersion of cubic gel particles to form a composition; and
applying said composition to said keratinous materials.

55. A process for making-up or caring for keratinous materials comprising:
combining a pulverulent phase and a binder phase, wherein said binder phase is an aqueous continuous phase containing a dispersion of phytanetriol-based cubic gel particles comprising:
(i) phytanetriol, wherein said phytanetriol is present in an amount ranging from 0.1% to 15% by weight relative to the total weight of the binder phase, and
(ii) at least one dispersing and stabilizing agent chosen from surface-active agents chosen from optionally saturated, linear and branched surface-active agents comprising fatty chains having from 8 to 22 carbon atoms, wherein said at least one dispersing and stabilizing agent is present in an amount ranging from 0. 1% to 3% by weight relative to the total weight of said binder phase and further wherein said surface-active agents are water-soluble at room temperature to form a composition; and
applying said composition to said keratinous materials.

56. A process for making-up or caring for keratinous materials comprising:
combining a pulverulent phase and a binder phase, wherein said binder phase is chosen from compositions comprising:
(1) an aqueous phase, wherein said aqueous phase is present in an amount ranging from 60% to 98% by weight relative to the total weight of said composition; and
(2) an oily phase, wherein said oily phase is present in an amount ranging from 2% to 40% by weight relative to the total weight of said composition, and further wherein said oily phase is dispersed in said aqueous phase and is stabilized with the aid of cubic gel particles, wherein said cubic gel particles comprise:
(i) at least one compound chosen from phytanetriol, N-2-alkoxycarbonyl derivatives of N-methylglucamine, and unsaturated fatty acid monoglycerides, wherein said at least one compound is present in an amount ranging from 0.1% to 15% by weight relative to the total weight of the composition; and
(ii) at least one dispersing and stabilizing agent chosen from surface-active agents chosen from optionally saturated, linear and branched surface-active agents comprising fatty chains having 8 to 22 carbon atoms, wherein said at least one dispersing and stabilizing agent is present in an amount ranging from 0.05% to 3% by weight relative to the total weight of said composition and further wherein said surface-active agents are water-soluble at room temperature, to form a composition; and
applying said composition to said keratinous materials.

57. A process for improving the dispersion of at least one pulverulent compound in a composition comprising a pulverulent phase and a binder phase comprising:
incorporating a dispersion of cubic gel particles into said binder phase; and
combining said binder phase and said pulverulent phase to form said composition.

58. A process for improving the moisturization of a composition comprising a pulverulent phase and a binder phase on keratinous materials comprising:
incorporating a dispersion of cubic gel particles into said binder phase;
combining said binder phase and said pulverulent phase to form said composition; and
applying said composition to said keratinous materials.

59. A make-up kit comprising a plurality of containers, wherein said plurality of containers comprises at least a first container and a second container, said first container comprises a composition comprising at least one dispersion of cubic gel particles and said second container comprises at least one pulverulent compound.

60. A process for making up keratin materials comprising:
a) mixing at least one pulverulent compound with an effective amount of a binder phase comprising a dispersion of cubic gel particles to form a homogeneous composition;
b) applying said homogeneous composition to said keratin materials.

61. A process according to claim 60, wherein said mixing is accomplished with a finger in the palm of the hand.

62. A process according to claim 60, wherein a relationship between said at least one pulverulent and said binder phase is a ratio, A/B, ranging from 100:1 to 1:1, wherein A is the mass in grams of said binder phase and B is the mass in grams of said at least one pulverulent compound.

63. A make-up composition according to claim 1, wherein said cubic gel particles ranges from 0.05 to 1 μm.

64. A make-up composition according to claim 1, wherein said cubic gel particles are less than or equal to 0.5 μm.

65. A make-up composition according to claim 1, wherein said binder phase has a pH ranging from 5 to 8.

66. A make-up composition according to claim 65, wherein said binder phase has a pH ranging from 6 to 7.

67. A make-up composition according to claim 1, wherein said binder phase comprises:
(1) an aqueous phase, wherein said aqueous phase is present in an amount ranging from 60% to 98% by weight relative to the total weight of said composition; and
(2) an oily phase, wherein said oily phase is present in an amount ranging from 2% to 40% by weight relative to the total weight of said composition, and further wherein said oily phase is dispersed in said aqueous phase and is stabilized with the aid of cubic gel particles, said cubic gel particles being formed from at least:
(i) at least one compound chosen from phytanetriol, N-2-alkoxycarbonyl derivatives of N-methylglucamine, and unsaturated fatty acid monoglycerides, wherein said at least one compound is present in an amount ranging from 0.1% to 15% by weight relative to the total weight of the composition; and
(ii) at least one dispersing and stabilizing agent chosen from surface-active agents chosen from optionally saturated, linear and branched surface-active agents comprising fatty chains having 8 to 22 carbon atoms, wherein said at least one dispersing and stabilizing agent is present in an amount ranging from 0.05% to 3% by weight relative to the total weight of said composition and further wherein said surface-active agents are water-soluble at room temperature.

68. A process for making-up or caring for keratinous materials comprising:
combining a pulverulent phase and a binder phase, wherein said binder phase is an aqueous continuous phase containing a dispersion of phytanetriol-based cubic gel particles formed from at least:
(i) phytanetriol, wherein said phytanetriol is present in an amount ranging from 0.1% to 15% by weight relative to the total weight of the binder phase, and
(ii) at least one dispersing and stabilizing agent chosen from surface-active agents chosen from optionally saturated, linear and branched surface-active agents comprising fatty chains having from 8 to 22 carbon atoms, wherein said at least one dispersing and stabilizing agent is present in an amount ranging from 0.1% to 3% by weight relative to the total weight of said binder phase and further wherein said surface-active agents are water-soluble at room temperature to form a composition; and
applying said composition to said keratinous materials.

69. A process for making-up or caring for keratinous materials comprising:
combining a pulverulent phase and a binder phase, wherein said binder phase is chosen from compositions comprising:
(1) an aqueous phase, wherein said aqueous phase is present in an amount ranging from 60% to 98% by weight relative to the total weight of said composition; and
(2) an oily phase, wherein said oily phase is present in an amount ranging from 2% to 40% by weight relative to the total weight of said composition, and further wherein said oily phase is dispersed in said aqueous phase and is stabilized with the aid of cubic gel particles, wherein said cubic gel particles are formed from at least:
(i) at least one compound chosen from phytanetriol, N-2-alkoxycarbonyl derivatives of N-methylglucamine, and unsaturated fatty acid monoglycerides, wherein said at least one compound is present in an amount ranging from 0.1% to 15% by weight relative to the total weight of the composition; and
(ii) at least one dispersing and stabilizing agent chosen from surface-active agents chosen from optionally saturated, linear and branched surface-active agents comprising fatty chains having 8 to 22 carbon atoms, wherein said at least one dispersing and stabilizing agent is present in an amount ranging from 0.05% to 3% by weight relative to the total weight of said composition and further wherein said surface-active agents are water-soluble at room temperature, to form a composition; and
applying said composition to said keratinous materials.
